SEQUENCE LISTING (81 sequences)

SEQ ID NO: 1 - protein, 199 aa, Artificial Sequence
  APEX Fragment 1
GKSYPTVSADYQDAVEKAKKRLGGFIAEKRCAPLMLRLAFHSAGTFDKRTKTGGPFGTIRYPAELAHSANSGLDIAVRLLEPLKAEFPILSYADFYQLAGVVAVEVTGGPKVPFHPGREDKPELPPEGRLPDPTKGSDHLRDVFGKAMGLTDQDIVALSGGHTLGAAHKERSGFEGPWTSNPLVFDNSYFTELLSGEKE

SEQ ID NO: 2 - protein, 177 aa, Artificial Sequence
  APEX C32S Fragment 1
GKSYPTVSADYQDAVEKAKKRLGGFIAEKRSAPLMLRLAFHSAGTFDKRTKTGGPFGTIRYPAELAHSANSGLDIAVRLLEPLKAEFPILSYADFYQLAGVVAVEVTGGPKVPFHPGREDKPELPPEGRLPDPTKGSDHLRDVFGKAMGLTDQDIVALSGGHTLGAAHKERSGFEGP

SEQ ID NO: 3 - protein, 62 aa, Artificial Sequence
  APEX Fragment 2
GLLQLPSDKALLSDPVFRPLVDKYAADEDAFFADYAEAHQKLSELGFADALQLPPLERLTLD

SEQ ID NO: 4 - protein, 213 aa, Artificial Sequence
  HRP Fragment 1
QLTPTFYDNSCPNVSNIVRDIIVNELRSDPRIAASILRLHFHDCFVNGCDASILLDNTTSFRTEKDAFGNANSARGFSVIDRMKAAVESACPGTVSCADLLTIAAQQSVTLAGGPSWRVPLGRRDSLQAFLDLANANLPAPFFTLPQLKDSFRNVGLNRSSDLVALSGGHTFGKSQCRFIMDRLYNFSNTGLPDPTLNTTYLQTLRGLCPLNG

SEQ ID NO: 5 - protein, 95 aa, Artificial Sequence
  HRP Fragment 2
NLSALVDFDLRTPTIFDNKYYVNLEEQKGLIQSDQELFSSPDATDTIPLVRSFANSTQTFFNAFVEAMDRMGNITPLTGTQGQIRRNCRVVNSNS

SEQ ID NO: 6 - protein, 179 aa, Artificial Sequence
  Luciferase Fragment 1
EDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYD

SEQ ID NO: 7 - protein, 157 aa, Artificial Sequence
  Luciferase Fragment 2
GPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 8 - protein, 157 aa, Artificial Sequence
  GFP Fragment 1
MASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNHNVLIMADKQ

SEQ ID NO: 9 - protein, 81 aa, Artificial Sequence
  GFP Fragment 2
KNGIKVNFKTRHNIEDGSVQLADHYQQNTPIGDGPVLLPDNHYLSTQSALSKDPNEKRDHMVLLEFVTAAGITHGMDELYN

SEQ ID NO: 10 - protein, 470 aa, Artificial Sequence
  Alkaline Phosphatase monomer
KQSTIALALLPLLFTPVTKARTPEMPVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 11 - protein, 587 aa, Artificial Sequence
  Glucose Oxidase monomer
YLPAQQIDVQSSLLSDPSKVAGKTYDYIIAGGGLTGLTVAAKLTENPKIKVLVIEKGFYESNDGAIIEDPNAYGQIFGTTVDQNYLTVPLINNRTNNIKAGKGLGGSTLINGDSWTRPDKVQIDSWEKVFGMEGWNWDNMFEYMKKAEAARTPTAAQLAAGHSFNATCHGTNGTVQSGARDNGQPWSPIMKALMNTVSALGVPVQQDFLCGHPRGVSMIMNNLDENQVRVDAARAWLLPNYQRSNLEILTGQMVGKVLFKQTASGPQAVGVNFGTNKAVNFDVFAKHEVLLAAGSAISPLILEYSGIGLKSVLDQANVTQLLDLPVGINMQDQTTTTVSSRASSAGAGQGQAVFFANFTETFGDYAPQARDLLNTKLDQWAEETVARGGFHNVTALKVQYENYRNWLLDEDVAFAELFMDTEGKINFDLWDLIPFTRGSVHILSSDPYLWQFANDPKFFLNEFDLLGQAAASKLARDLTSQGAMKEYFAGETLPGYNLVQNATLSQWSDYVLQNFRPNWHAVSSCSMMSRELGGVVDATAKVYGTQGLRVIDGSIPPTQVSSHVMTIFYGMALKVADAILDDYAKSA

SEQ ID NO: 12 - protein, 35 aa, Artificial Sequence
  Leucine Zipper Monera 1
KCEALEGKLEALEGKAEALEGKLEALEGKLEALEG

SEQ ID NO: 13 - protein, 35 aa, Artificial Sequence
  Leucine Zipper Monera 2
ELAELKGELAELKGELAEAKGELAELKGELAECKG

SEQ ID NO: 14 - protein, 29 aa, Artificial Sequence
  Leucine Zipper Gosh 1
LLKKELQLNKKELLQLKWELQLLKKELLQ

SEQ ID NO: 15 - protein, 29 aa, Artificial Sequence
  Leucine Zipper Gosh 2
QLEKKLQLLEKKLLQLEWKNQLLEKKLLQ

SEQ ID NO: 16 - protein, 30 aa, Artificial Sequence
  Leucine Zipper Acid A1
AQLEKELQALEKELAQLEWENQALEKELAQ

SEQ ID NO: 17 - protein, 30 aa, Artificial Sequence
  Leucine Zipper Base A1
AQLKKKLQANKKKLAQLKWKLQALKKKLAQ

SEQ ID NO: 18 - protein, 30 aa, Artificial Sequence
  Leucine Zipper Acid KG
AQLEKELQALEKKLAQLEWENQALEKELAQ

SEQ ID NO: 19 - protein, 30 aa, Artificial Sequence
  Leucine Zipper Base EG
AQLKKKLQANKKELAQLKWKLQALKKKLAQ

SEQ ID NO: 20 - protein, 251 aa, Artificial Sequence
  scFv Construct B38_VH-VL
MHHHHHHMEVQLVESGGGLVQPGGSLRLSCAASGFIVSSNYMSWVRQAPGKGLEWVSVIYSGGSTYYADSVKGRFTISRHNSKNTLYLQMNSLRAEDTAVYYCAREAYGMDVWGQGTTVTVSSGGGGSGGGGSGGGGSDIVMTQSPSFLSASVGDRVTITCRASQGISSYLAWYQQKPGKAPKLLIYAASTLQSGVPSRFSGSGSGTEFTLTISSLQPEDFATYYCQQLNSYPPYTFGQGTKLEIKGGGGS

SEQ ID NO: 21 - protein, 246 aa, Artificial Sequence
  scFv Construct B38_VL-VH
MHHHHHHMDIVMTQSPSFLSASVGDRVTITCRASQGISSYLAWYQQKPGKAPKLLIYAASTLQSGVPSRFSGSGSGTEFTLTISSLQPEDFATYYCQQLNSYPPYTFGQGTKLEIKGGGGSGGGGSGGGGSEVQLVESGGGLVQPGGSLRLSCAASGFIVSSNYMSWVRQAPGKGLEWVSVIYSGGSTYYADSVKGRFTISRHNSKNTLYLQMNSLRAEDTAVYYCAREAYGMDVWGQGTTVTVSS

SEQ ID NO: 22 - protein, 267 aa, Artificial Sequence
  scFv Construct H4_VH-VL
MHHHHHHMQVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARVPYCSSTSCHRDWYFDLWGRGTLVTVSSGGGGSGGGGSGGGGSDIQMTQSPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPLTFGGGTKVEIKGGGGS

SEQ ID NO: 23 - protein, 267 aa, Artificial Sequence
  scFv Construct H4_VL-VH
MHHHHHHMDIQMTQSPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPLTFGGGTKVEIKSGGGGSGGGGSGGGGSQVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARVPYCSSTSCHRDWYFDLWGRGTLVTVSGGGGS

SEQ ID NO: 24 - protein, 336 aa, Artificial Sequence
  AP-M1Lock-Acid-His
MGKSYPTVSADYQDAVEKAKKRLGGFIAEKRCAPLMLRLAFHSAGTFDKRTKTGGPFGTIRYPAELAHSANSGLDIAVRLLEPLKAEFPILSYADFYQLAGVVAVEVTGGPKVPFHPGREDKPELPPEGRLPDPTKGSDHLRDVFGKAMGLTDQDIVALSGGHTLGAAHKERSGFEGPWTSNPLVFDNSYFTELLSGEKEGGGGSGGGGSGGGGSASLAQCEKKLQALEKKAAQLEWKLQALEKKLAQLGGGGSGGGGSGGGGSASLAQLEKELQALEKKLAQLEWENQALEKELAQLGGGGSGGGGSGGGGSGGGGSGGGGSGGGGSASHHHHHH

SEQ ID NO: 25 - protein, 199 aa, Artificial Sequence
  EX-M2Lock-Base-His
MHHHHHHGGGGSGGGGSGGGGSGGGGSGGGGSGGGGSASLAQLKKKLQANKKELAQLKWKLQALKKKLAQLGGGGSGGGGSGGGGSASLAQLKKELQALKWELAQAKKELQALKKECAQLGGGGSGGGGSGGGGSASGLLQLPSDKALLSDPVFRPLVDKYAADEDAFFADYAEAHQKLSELGFADALQLPPLERLTLD

SEQ ID NO: 26 - protein, 336 aa, Artificial Sequence
  APC32S-M1Lock-Acid-His
MGKSYPTVSADYQDAVEKAKKRLGGFIAEKRSAPLMLRLAFHSAGTFDKRTKTGGPFGTIRYPAELAHSANSGLDIAVRLLEPLKAEFPILSYADFYQLAGVVAVEVTGGPKVPFHPGREDKPELPPEGRLPDPTKGSDHLRDVFGKAMGLTDQDIVALSGGHTLGAAHKERSGFEGPWTSNPLVFDNSYFTELLSGEKEGGGGSGGGGSGGGGSASLAQCEKKLQALEKKAAQLEWKLQALEKKLAQLGGGGSGGGGSGGGGSASLAQLEKELQALEKKLAQLEWENQALEKELAQLGGGGSGGGGSGGGGSGGGGSGGGGSGGGGSASHHHHHH

SEQ ID NO: 27 - protein, 336 aa, Artificial Sequence
  AP-M1Lock-Base-His
MGKSYPTVSADYQDAVEKAKKRLGGFIAEKRCAPLMLRLAFHSAGTFDKRTKTGGPFGTIRYPAELAHSANSGLDIAVRLLEPLKAEFPILSYADFYQLAGVVAVEVTGGPKVPFHPGREDKPELPPEGRLPDPTKGSDHLRDVFGKAMGLTDQDIVALSGGHTLGAAHKERSGFEGPWTSNPLVFDNSYFTELLSGEKEGGGGSGGGGSGGGGSASLAQCEKKLQALEKKAAQLEWKLQALEKKLAQLGGGGSGGGGSGGGGSASLAQLKKKLQANKKELAQLKWKLQALKKKLAQLGGGGSGGGGSGGGGSGGGGSGGGGSGGGGSASHHHHHH

SEQ ID NO: 28 - protein, 199 aa, Artificial Sequence
  EX-M2Lock-Acid-His
MHHHHHHGGGGSGGGGSGGGGSGGGGSGGGGSGGGGSASLAQLEKELQALEKKLAQLEWENQALEKELAQLGGGGSGGGGSGGGGSASLAQLKKELQALKWELAQAKKELQALKKECAQLGGGGSGGGGSGGGGSASGLLQLPSDKALLSDPVFRPLVDKYAADEDAFFADYAEAHQKLSELGFADALQLPPLERLTLD

SEQ ID NO: 29 - protein, 336 aa, Artificial Sequence
  APC32S-M1Lock-Base-His
MGKSYPTVSADYQDAVEKAKKRLGGFIAEKRSAPLMLRLAFHSAGTFDKRTKTGGPFGTIRYPAELAHSANSGLDIAVRLLEPLKAEFPILSYADFYQLAGVVAVEVTGGPKVPFHPGREDKPELPPEGRLPDPTKGSDHLRDVFGKAMGLTDQDIVALSGGHTLGAAHKERSGFEGPWTSNPLVFDNSYFTELLSGEKEGGGGSGGGGSGGGGSASLAQCEKKLQALEKKAAQLEWKLQALEKKLAQLGGGGSGGGGSGGGGSASLAQLKKKLQANKKELAQLKWKLQALKKKLAQLGGGGSGGGGSGGGGSGGGGSGGGGSGGGGSASHHHHHH

SEQ ID NO: 30 - protein, 336 aa, Artificial Sequence
  AP-M1Latch-Acid-His
MGKSYPTVSADYQDAVEKAKKRLGGFIAEKRCAPLMLRLAFHSAGTFDKRTKTGGPFGTIRYPAELAHSANSGLDIAVRLLEPLKAEFPILSYADFYQLAGVVAVEVTGGPKVPFHPGREDKPELPPEGRLPDPTKGSDHLRDVFGKAMGLTDQDIVALSGGHTLGAAHKERSGFEGPWTSNPLVFDNSYFTELLSGEKEGGGGSGGGGSGGGGSASLAQSEKKLQALEKKAAQLEWKLQALEKKLAQLGGGGSGGGGSGGGGSASLAQLEKELQALEKKLAQLEWENQALEKELAQLGGGGSGGGGSGGGGSGGGGSGGGGSGGGGSASHHHHHH

SEQ ID NO: 31 - protein, 199 aa, Artificial Sequence
  EX-M2Latch-Base-His
MHHHHHHGGGGSGGGGSGGGGSGGGGSGGGGSGGGGSASLAQLKKKLQANKKELAQLKWKLQALKKKLAQLGGGGSGGGGSGGGGSASLAQLKKELQALKWELAQAKKELQALKKESAQLGGGGSGGGGSGGGGSASGLLQLPSDKALLSDPVFRPLVDKYAADEDAFFADYAEAHQKLSELGFADALQLPPLERLTLD

SEQ ID NO: 32 - protein, 336 aa, Artificial Sequence
  APC32S-M1Latch-Acid-His
MGKSYPTVSADYQDAVEKAKKRLGGFIAEKRSAPLMLRLAFHSAGTFDKRTKTGGPFGTIRYPAELAHSANSGLDIAVRLLEPLKAEFPILSYADFYQLAGVVAVEVTGGPKVPFHPGREDKPELPPEGRLPDPTKGSDHLRDVFGKAMGLTDQDIVALSGGHTLGAAHKERSGFEGPWTSNPLVFDNSYFTELLSGEKEGGGGSGGGGSGGGGSASLAQSEKKLQALEKKAAQLEWKLQALEKKLAQLGGGGSGGGGSGGGGSASLAQLEKELQALEKKLAQLEWENQALEKELAQLGGGGSGGGGSGGGGSGGGGSGGGGSGGGGSASHHHHHH

SEQ ID NO: 33 - protein, 336 aa, Artificial Sequence
  AP-M1Latch-Base-His
MGKSYPTVSADYQDAVEKAKKRLGGFIAEKRCAPLMLRLAFHSAGTFDKRTKTGGPFGTIRYPAELAHSANSGLDIAVRLLEPLKAEFPILSYADFYQLAGVVAVEVTGGPKVPFHPGREDKPELPPEGRLPDPTKGSDHLRDVFGKAMGLTDQDIVALSGGHTLGAAHKERSGFEGPWTSNPLVFDNSYFTELLSGEKEGGGGSGGGGSGGGGSASLAQSEKKLQALEKKAAQLEWKLQALEKKLAQLGGGGSGGGGSGGGGSASLAQLKKKLQANKKELAQLKWKLQALKKKLAQLGGGGSGGGGSGGGGSGGGGSGGGGSGGGGSASHHHHHH

SEQ ID NO: 34 - protein, 199 aa, Artificial Sequence
  AP-M2Latch-Acid-His
MHHHHHHGGGGSGGGGSGGGGSGGGGSGGGGSGGGGSASLAQLEKELQALEKKLAQLEWENQALEKELAQLGGGGSGGGGSGGGGSASLAQLKKELQALKWELAQAKKELQALKKESAQLGGGGSGGGGSGGGGSASGLLQLPSDKALLSDPVFRPLVDKYAADEDAFFADYAEAHQKLSELGFADALQLPPLERLTLD

SEQ ID NO: 35 - protein, 336 aa, Artificial Sequence
  APC32S-M1Latch-Base-His
MGKSYPTVSADYQDAVEKAKKRLGGFIAEKRSAPLMLRLAFHSAGTFDKRTKTGGPFGTIRYPAELAHSANSGLDIAVRLLEPLKAEFPILSYADFYQLAGVVAVEVTGGPKVPFHPGREDKPELPPEGRLPDPTKGSDHLRDVFGKAMGLTDQDIVALSGGHTLGAAHKERSGFEGPWTSNPLVFDNSYFTELLSGEKEGGGGSGGGGSGGGGSASLAQSEKKLQALEKKAAQLEWKLQALEKKLAQLGGGGSGGGGSGGGGSASLAQLKKKLQANKKELAQLKWKLQALKKKLAQLGGGGSGGGGSGGGGSGGGGSGGGGSGGGGSASHHHHHH

SEQ ID NO: 36 - protein, 609 aa, Artificial Sequence
  AP-H4-Avitag-His
MGKSYPTVSADYQDAVEKAKKRLGGFIAEKRCAPLMLRLAFHSAGTFDKRTKTGGPFGTIRYPAELAHSANSGLDIAVRLLEPLKAEFPILSYADFYQLAGVVAVEVTGGPKVPFHPGREDKPELPPEGRLPDPTKGSDHLRDVFGKAMGLTDQDIVALSGGHTLGAAHKERSGFEGPWTSNPLVFDNSYFTELLSGEKEGGGGSGGGGSGGGGSASLAQSEKKLQALEKKAAQLEWKLQALEKKLAQLGGGGSGGGGSGGGGSASLAQLKKKLQANKKELAQLKWKLQALKKKLAQLGGGGSGGGGSGGGGSGGGGSGGGGSMDIQMTQSPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPLTFGGGTKVEIKSGGGGSGGGGSGGGGSQVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARVPYCSSTSCHRDWYFDLWGRGTLVTVSGGGGSGLNDIFEAQKIEWHEAAALEHHHHHH

SEQ ID NO: 37 - protein, 438 aa, Artificial Sequence
  B38VHVL-EX-His
MHHHHHHMEVQLVESGGGLVQPGGSLRLSCAASGFIVSSNYMSWVRQAPGKGLEWVSVIYSGGSTYYADSVKGRFTISRHNSKNTLYLQMNSLRAEDTAVYYCAREAYGMDVWGQGTTVTVSSGGGGSGGGGSGGGGSDIVMTQSPSFLSASVGDRVTITCRASQGISSYLAWYQQKPGKAPKLLIYAASTLQSGVPSRFSGSGSGTEFTLTISSLQPEDFATYYCQQLNSYPPYTFGQGTKLEIKGGGGSGGGGSGGGGSGGGGSGGGGSGGGGSASLAQLEKELQALEKKLAQLEWENQALEKELAQLGGGGSGGGGSGGGGSASLAQLKKELQALKWELAQAKKELQALKKESAQLGGGGSGGGGSGGGGSASGLLQLPSDKALLSDPVFRPLVDKYAADEDAFFADYAEAHQKLSELGFADALQLPPLERLTLD

SEQ ID NO: 38 - protein, 472 aa, Artificial Sequence
  Split Luc LL 1
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMGGGGSGGGGSGGGGSGGGGSQLKKKLQALKKKLAQLKWKLQAGGGGSGGGGSALEKELAQLEWELQALEKELAQ

SEQ ID NO: 39 - protein, 232 aa, Artificial Sequence
  Split Luc LL 2
MALKKKLAQLKWKLQALKKKLAQGGGGSGGGGSQLEKELQALEKELAQLEWELQAGGGGSGGGGSGGGGSGGGGSGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 40 - protein, 559 aa, Artificial Sequence
  Alkaline Phosphatase Conjugated
MKQSTIALALLPLLFTPVTKARTPEMPVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLKGGGGSGGGGSGGGGSGGGGSQLEKELQALEKKLAQLKWKNQALKKELAQGGGGSGGGGSQLKKELQALEKELAQLEWKLQANKKKLAQ

SEQ ID NO: 41 - protein, 676 aa, Artificial Sequence
  Gox Conjugated
MQLEKENQALEKKLAQLKWKLQALKKELAQGGGGSGGGGSQLKKELQANEKELAQLEWKLQALKKKLAQGGGGSGGGGSGGGGSGGGGSYLPAQQIDVQSSLLSDPSKVAGKTYDYIIAGGGLTGLTVAAKLTENPKIKVLVIEKGFYESNDGAIIEDPNAYGQIFGTTVDQNYLTVPLINNRTNNIKAGKGLGGSTLINGDSWTRPDKVQIDSWEKVFGMEGWNWDNMFEYMKKAEAARTPTAAQLAAGHSFNATCHGTNGTVQSGARDNGQPWSPIMKALMNTVSALGVPVQQDFLCGHPRGVSMIMNNLDENQVRVDAARAWLLPNYQRSNLEILTGQMVGKVLFKQTASGPQAVGVNFGTNKAVNFDVFAKHEVLLAAGSAISPLILEYSGIGLKSVLDQANVTQLLDLPVGINMQDQTTTTVSSRASSAGAGQGQAVFFANFTETFGDYAPQARDLLNTKLDQWAEETVARGGFHNVTALKVQYENYRNWLLDEDVAFAELFMDTEGKINFDLWDLIPFTRGSVHILSSDPYLWQFANDPKFFLNEFDLLGQAAASKLARDLTSQGAMKEYFAGETLPGYNLVQNATLSQWSDYVLQNFRPNWHAVSSCSMMSRELGGVVDATAKVYGTQGLRVIDGSIPPTQVSSHVMTIFYGMALKVADAILDDYAKSA

SEQ ID NO: 42 - protein, 81 aa, Artificial Sequence
  M1Lock
LAQCEKKLQALEKKAAQLEWKLQALEKKLAQLGGGGSGGGGSGGGGSASLAQLEKELQALEKKLAQLEWENQALEKELAQL

SEQ ID NO: 43 - protein, 81 aa, Artificial Sequence
  M2Lock
LAQLKKKLQANKKELAQLKWKLQALKKKLAQLGGGGSGGGGSGGGGSASLAQLKKELQALKWELAQAKKELQALKKECAQL

SEQ ID NO: 44 - protein, 81 aa, Artificial Sequence
  M2Latch
LAQLEKELQALEKKLAQLEWENQALEKELAQLGGGGSGGGGSGGGGSASLAQLKKELQALKWELAQAKKELQALKKESAQL

SEQ ID NO: 45 - protein, 81 aa, Artificial Sequence
  M1Latch
LAQSEKKLQALEKKAAQLEWKLQALEKKLAQLGGGGSGGGGSGGGGSASLAQLKKKLQANKKELAQLKWKLQALKKKLAQL

SEQ ID NO: 46 - protein, 68 aa, Artificial Sequence
  LL4Heptad0H0S-1
QLEKELQALEKKLAQLEWENQALEKELAQGGGGSGGGGSQLKKKLQANKKELAQLKWKLQALKKKLAQ

SEQ ID NO: 47 - protein, 68 aa, Artificial Sequence
  LL4Heptad1H0S-1
QLEKELQALEKKLAQLKWKNQALKKELAQGGGGSGGGGSQLKKELQALEKELAQLEWKLQALKKKLAQ

SEQ ID NO: 48 - protein, 68 aa, Artificial Sequence
  LL4Heptad2H0S-1
QLEKELQALEKKLAQLKWKNQALKKELAQGGGGSGGGGSQLKKELQALEKELAQLEWKLQANKKKLAQ

SEQ ID NO: 49 - protein, 68 aa, Artificial Sequence
  LL4Heptad0H2S-1
QLEKELQALEKKLAQLEWKNQALKKELAQGGGGSGGGGSQLKKKLQANKKELAQLKWKLQALKKKLAQ

SEQ ID NO: 50 - protein, 68 aa, Artificial Sequence
  LL4Heptad1H2S-1
QLEKELQALEKKLAQLEWKNQALKKELAQGGGGSGGGGSQLKKKLQALKKELAQLKWKLQALKKKLAQ

SEQ ID NO: 51 - protein, 68 aa, Artificial Sequence
  LL4Heptad2H2S-1
QLEKELQALEKKLAQLEWKNQALKKELAQGGGGSGGGGSQLKKKLQALKKELAQLKWKLQANKKKLAQ

SEQ ID NO: 52 - protein, 68 aa, Artificial Sequence; identical to SEQ ID NO: 46
  LL4Heptad0H0S-2

SEQ ID NO: 53 - protein, 68 aa, Artificial Sequence
  LL4Heptad1H0S-2
QLEKELQALEKKLAQLKWKLQALKKELAQGGGGSGGGGSQLKKELQANEKELAQLEWKLQALKKKLAQ

SEQ ID NO: 54 - protein, 68 aa, Artificial Sequence
  LL4Heptad2H0S-2
QLEKENQALEKKLAQLKWKLQALKKELAQGGGGSGGGGSQLKKELQANEKELAQLEWKLQALKKKLAQ

SEQ ID NO: 55 - protein, 68 aa, Artificial Sequence
  LL4Heptad0H2S-2
QLEKELQALEKKLAQLEWENQALEKELAQGGGGSGGGGSQLKKELQANEKELAQLKWKLQALKKKLAQ

SEQ ID NO: 56 - protein, 68 aa, Artificial Sequence
  LL4Heptad1H2S-2
QLEKELQALEKKLAQLEWELQALEKELAQGGGGSGGGGSQLKKELQANEKELAQLKWKLQALKKKLAQ

SEQ ID NO: 57 - protein, 68 aa, Artificial Sequence
  LL4Heptad2H2S-2
QLEKENQALEKKLAQLEWELQALEKELAQGGGGSGGGGSQLKKELQANEKELAQLKWKLQALKKKLAQ

SEQ ID NO: 58 - protein, 54 aa, Artificial Sequence
  LL3Heptad0H0S0N-1
QLKKKLQALKKKLAQLKWKLQAGGGGSGGGGSALEKELAQLEWELQALEKELAQ

SEQ ID NO: 59 - protein, 54 aa, Artificial Sequence
  LL3Heptad0H0S2N-1
QNKKKLQALKKKLAQLKWKLQAGGGGSGGGGSALEKELAQLEWELQALEKENAQ

SEQ ID NO: 60 - protein, 54 aa, Artificial Sequence
  LL3Heptad1H0S-1
QLKKKLQALKKKLAQLKWKNQAGGGGSGGGGSALEKELAQLEWELQALEKELAQ

SEQ ID NO: 61 - protein, 54 aa, Artificial Sequence
  LL3Heptad2H0S-1
QLKKKLQALKKKLAQLKWKNQAGGGGSGGGGSALEKENAQLEWELQALEKELAQ

SEQ ID NO: 62 - protein, 54 aa, Artificial Sequence
  LL3Heptad0H2S0N-1
QLEKKLQALKKELAQLKWKLQAGGGGSGGGGSALEKELAQLKWELQALEKELAQ

SEQ ID NO: 63 - protein, 54 aa, Artificial Sequence
  LL3Heptad0H2S2N-1
QNEKKLQALKKELAQLKWKLQAGGGGSGGGGSALEKELAQLKWELQALEKENAQ

SEQ ID NO: 64 - protein, 54 aa, Artificial Sequence
  LL3Heptad1H2S-1
QLEKKLQALKKELAQLKWKLQAGGGGSGGGGSALEKELAQLKWELQALEKENAQ

SEQ ID NO: 65 - protein, 54 aa, Artificial Sequence
  LL3Heptad2H2S-1
QNEKKLQALKKELAQLKWKNQAGGGGSGGGGSALEKENAQLKWELQALEKENAQ

SEQ ID NO: 66 - protein, 54 aa, Artificial Sequence
  LL3Heptad0H0S0N-2
ALKKKLAQLKWKLQALKKKLAQGGGGSGGGGSQLEKELQALEKELAQLEWELQA

SEQ ID NO: 67 - protein, 54 aa, Artificial Sequence
  LL3Heptad0H0S2N-2
ANKKKLAQLKWKLQALKKKLAQGGGGSGGGGSQLEKELQALEKELAQLEWENQA

SEQ ID NO: 68 - protein, 54 aa, Artificial Sequence
  LL3Heptad1H0S-2
ALKKKLAQLKWKLQALKKKLAQGGGGSGGGGSQNEKELQALEKELAQLEWELQA

SEQ ID NO: 69 - protein, 54 aa, Artificial Sequence
  LL3Heptad2H0S-2
ALKKKLAQLKWKLQANKKKLAQGGGGSGGGGSQNEKELQALEKELAQLEWELQA

SEQ ID NO: 70 - protein, 54 aa, Artificial Sequence
  LL3Heptad0H2S0N-2
ALKKKLAQLKWKLQALEKKLAQGGGGSGGGGSQLKKKLQALEKELAQLEWELQA

SEQ ID NO: 71 - protein, 54 aa, Artificial Sequence
  LL3Heptad0H2S2N-2
ANKKKLAQLKWKLQALEKKLAQGGGGSGGGGSQLKKKLQALEKELAQLEWENQA

SEQ ID NO: 72 - protein, 54 aa, Artificial Sequence
  LL3Heptad1H2S-2
ANKKKLAQLKWKLQALEKKLAQGGGGSGGGGSQLKKKLQALEKELAQLEWELQA

SEQ ID NO: 73 - protein, 54 aa, Artificial Sequence
  LL3Heptad2H2S-2
ANKKKLAQLKWKLQANEKKLAQGGGGSGGGGSQNKKKLQALEKELAQLEWENQA

SEQ ID NO: 74 - protein, 244 aa, Artificial Sequence
  ScFv Hui-18
QVQLQQSGAELVRPGTSVKVSCKASGYAFTNHLIEWVNQRPGQGLEWIGVINPGSGGTKYNEKFKGKATLTADKSSSTAYMQLSRLTSDDSAVYFCARSSEFITTVAADYWGQGTTGGGGSGGGGSGGGGSGGGGSDIVMTQSQKFMSTSVGDRVSITCKASQNVRTAVAWYQQRPGQSPKALIYLASNRHTGVPDRFTGSGSGTDFTLTITNVQSEDLADYFCLQHWNYPLTFGSGTKLEIKR

SEQ ID NO: 75 - protein, 242 aa, Artificial Sequence
  Insulin ScFv Oxi-5
EVQLVESGGGLVKPGGSLKLSCTASGFAFSDYDMSWVRQTPEKRLEWVAFISNGGYSTYYPDTVKGRFTISRDNAENTLYLQMSSLKSEDTAIYYCARQGLRYFDYWGLGTTLTVSGGGGSGGGGSGGGGSGGGGSDIQMTQSPSSLSASLGGRVTITCKASQDINKYLAWYQHKPGKGPRLLIHYTSTLQPGIPSRFSGSGSGRDYSFSISNLEPEDVATYYCLQYDSLLSFGAGTKLELK

SEQ ID NO: 76 - protein, 250 aa, Artificial Sequence
  Insulin ScFv CGCG7
EVQLQQSGPELVKPGASVKISCKTSGYTFTEYTMHWVKQSHGKSLEWIGGINPNNGGSNYNQKFKGKATLTVDKSSSTAYMELRSLTSEDSAVYYCARFGARYYGSSYGSWYFDVWGAGTTVTVSGGGGSGGGGSGGGGSGGGGSDIVLTQSPATLSVTPGDSVSLSCRASQSISNNLHWYQQKSHESPRLLIKYASQSISGIPSRFSGSGTDFTLSINSVETEDFGMYFCQQSNSWPHTFGSGTKLEIK

SEQ ID NO: 77 - protein, 257 aa, Artificial Sequence
  Insulin ScFv HB125
QIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKGGGGSGGGGSGGGGSGGGGSQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGS

SEQ ID NO: 78 - protein, 538 aa, Artificial Sequence
  COVID-19 Spike Protein Biosensor 1; Split APEX Fragment 1; COVID-19 Spike Protein ScFV 1
MGKSYPTVSADYQDAVEKAKKRLGGFIAEKRCAPLMLRLAFHSAGTFDKRTKTGGPFGTIRYPAELAHSANSGLDIAVRLLEPLKAEFPILSYADFYQLAGVVAVEVTGGPKVPFHPGREDKPELPPEGRLPDPTKGSDHLRDVFGKAMGLTDQDIVALSGGHTLGAAHKERSGFEGPWTSNPLVFDNSYFTELLSGEKEGGGGSGGGGSGGGGSGGGGSQLEKKLQALKKELAQLKWKLQAGGGGSGGGGSALEKELAQLKWELQALEKENAQGGGGSGGGGSGGGGSGGGGSMEVQLVESGGGLVQPGGSLRLSCAASGFIVSSNYMSWVRQAPGKGLEWVSVIYSGGSTYYADSVKGRFTISRHNSKNTLYLQMNSLRAEDTAVYYCAREAYGMDVWGQGTTVTVSSGGGGSGGGGSGGGGSDIVMTQSPSFLSASVGDRVTITCRASQGISSYLAWYQQKPGKAPKLLIYAASTLQSGVPSRFSGSGSGTEFTLTISSLQPEDFATYYCQQLNSYPPYTFGQGTKLEIKGGGGS

SEQ ID NO: 79 - protein, 395 aa, Artificial Sequence
  COVID-19 Spike Protein Biosensor 2; COVID-19 Spike Protein ScFV 2; Split APEX Fragment 2
MDIVMTQSPSFLSASVGDRVTITCRASQGISSYLAWYQQKPGKAPKLLIYAASTLQSGVPSRFSGSGSGTEFTLTISSLQPEDFATYYCQQLNSYPPYTFGQGTKLEIKGGGGSGGGGSGGGGSEVQLVESGGGLVQPGGSLRLSCAASGFIVSSNYMSWVRQAPGKGLEWVSVIYSGGSTYYADSVKGRFTISRHNSKNTLYLQMNSLRAEDTAVYYCAREAYGMDVWGQGTTVTVSSGGGGSGGGGSGGGGSGGGGSANKKKLAQLKWKLQALEKKLAQGGGGSGGGGSQLKKKLQALEKELAQLEWELQAGGGGSGGGGSGGGGSGGGGSGLLQLPSDKALLSDPVFRPLVDKYAADEDAFFADYAEAHQKLSELGFADALQLPPLERLTLD

SEQ ID NO: 80 - protein, 509 aa, Artificial Sequence
  Insulin Biosensor 1; Split GFP Fragment 1; COVID-19 Spike Protein ScFV 1
MASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNHNVLIMADKQGGGGSGGGGSGGGGSGGGGSQLEKELQALEKKLAQLEWKNQALKKELAQGGGGSGGGGSQLKKKLQANKKELAQLKWKLQALKKKLAQGGGGSGGGGSGGGGSGGGGSQVQLQQSGAELVRPGTSVKVSCKASGYAFTNHLIEWVNQRPGQGLEWIGVINPGSGGTKYNEKFKGKATLTADKSSSTAYMQLSRLTSDDSAVYFCARSSEFITTVAADYWGQGTTGGGGSGGGGSGGGGSGGGGSDIVMTQSQKFMSTSVGDRVSITCKASQNVRTAVAWYQQRPGQSPKALIYLASNRHTGVPDRFTGSGSGTDFTLTITNVQSEDLADYFCLQHWNYPLTFGSGTKLEIKR

SEQ ID NO: 81 - protein, 432 aa, Artificial Sequence
  Insulin Biosensor 2; Insulin ScFV 2; Split GFP Fragment 2
MEVQLVESGGGLVKPGGSLKLSCTASGFAFSDYDMSWVRQTPEKRLEWVAFISNGGYSTYYPDTVKGRFTISRDNAENTLYLQMSSLKSEDTAIYYCARQGLRYFDYWGLGTTLTVSGGGGSGGGGSGGGGSGGGGSDIQMTQSPSSLSASLGGRVTITCKASQDINKYLAWYQHKPGKGPRLLIHYTSTLQPGIPSRFSGSGSGRDYSFSISNLEPEDVATYYCLQYDSLLSFGAGTKLELKGGGGSGGGGSGGGGSGGGGSQLEKELQALEKKLAQLEWENQALEKELAQGGGGSGGGGSQLKKELQANEKELAQLKWKLQALKKKLAQGGGGSGGGGSGGGGSGGGGSKNGIKVNFKTRHNIEDGSVQLADHYQQNTPIGDGPVLLPDNHYLSTQSALSKDPNEKRDHMVLLEFVTAAGITHGMDELYN

(19) United States
(12) Patent Application Publication
(10) Pub. No.: US 2022/0283151 A1
(43) Pub. Date: Sep. 8, 2022

(54) METHOD FOR CONTROLLING PROTEIN DIMERIZATION USING AN INTRAMOLECULAR TO INTERMOLECULAR CONFORMATIONAL SWITCH

(71) Applicant: Brigham Young University, Provo, UT (US)

(72) Inventor: Jonathon T. HILL, Provo, UT (US)

(21) Appl. No.: 17/653,643

(22) Filed: Mar. 4, 2022

Related U.S. Application Data
(60) Provisional application No. 63/200,428, filed on Mar. 5, 2021.

(51) Int. Cl. G01N 33/535; C07K 14/00; G01N 33/531

(52) U.S. Cl. CPC G01N 33/535; C07K 14/001; G01N 33/531

(57) ABSTRACT

A system for regulating protein dimerization kinetics is provided herein. The system includes a first polypeptide comprising a first alpha helix forming amino acid sequence configured to bind a second alpha helix forming amino acid sequence linked by a first flexible linker peptide; and a second polypeptide comprising a third alpha helix forming amino acid sequence configured to bind a fourth alpha helix forming amino acid sequence linked by a second flexible linker peptide. The first alpha helix forming amino acid sequence and the third alpha helix forming amino acid sequence are configured to form a coiled coil. The second alpha helix forming amino acid sequence and the fourth alpha helix forming amino acid sequence are configured to form a coiled coil.

### CROSS-REFERENCE TO RELATED APPLICATIONS

This application claims the benefit of U.S. provisional application 63/200,428 filed on Mar. 5, 2021, the disclosure of which is hereby incorporated in its entirety by reference herein.

### REFERENCE TO SEQUENCE LISTING

A sequence listing entitled “Sequence_Listing_354095.txt” is an ASCII text file and is incorporated herein by reference in its entirety. The text file was created on Mar. 5, 2021 and is 134 KB in size.

### TECHNICAL FIELD

The disclosure generally relates to compositions, polypeptides, methods, and systems for controlling protein dimerization or oligomerization of biosensors using intramolecular to intermolecular conformational switches.

### BACKGROUND

The rapid, inexpensive, and sensitive detection of analytes in biological or environmental samples would greatly improve health and safety. For example, the detection of viral or bacterial infections and physiological biomarkers at home or the point of care would make diagnosis more accurate and rapid, while limiting exposure to others. Rapid and inexpensive tests would also allow frequent testing of water supplies, restaurant surfaces, and other potential sources of exposure to further decrease the spread of existing or emerging diseases. However, current methods are limited due to the time, expertise, and often special equipment they require, leading to high costs and slow turnaround times.

One proposed method to simplify these assays is to create a split enzyme or multimeric protein complex that is reconstituted upon analyte binding. However, these methods currently have limited sensitivity due the need for continuous binding to the analyte for enzymatic activity, which prevents direct signal amplification. Thus, the system must be designed to favor enzyme formation as much as possible, but this can lead to the non-specific reconstitution of the enzyme in the absence of the analyte, resulting in a high number of false positives. To address these issues, complex mechanisms to separate the solution into various components or preprocessing steps are necessary, but this adds cost and complexity to the system.

### BRIEF SUMMARY

A system for regulating protein dimerization kinetics is provided. The system includes a first polypeptide comprising a first alpha helix forming amino acid sequence configured to bind a second alpha helix forming amino acid sequence linked by a first flexible linker peptide; and a second polypeptide comprising a third alpha helix forming amino acid sequence configured to bind a fourth alpha helix forming amino acid sequence linked by a second flexible linker peptide. The first alpha helix forming amino acid sequence and the third alpha helix forming amino acid sequence are configured to form a coiled coil. The second alpha helix forming amino acid sequence and the fourth alpha helix forming amino acid sequence are configured to form a coiled coil.

A biosensor is provided herein. The biosensor includes a first polypeptide comprising a first alpha helix forming amino acid sequence configured to bind a second alpha helix forming amino acid sequence linked by a first flexible linker peptide; a second polypeptide comprising a third alpha helix forming amino acid sequence configured to bind a fourth alpha helix forming amino acid sequence linked by a second flexible linker peptide; and a first signal generation component operably linked to the first polypeptide and a second signal generation component operably linked to the second polypeptide and a first analyte binding component operably linked to the first polypeptide and a second analyte binding component operably linked to the second polypeptide configured to promote formation of a dimer upon analyte binding that will persist after dissociation from the analyte. The first alpha helix forming amino acid sequence and the third alpha helix forming amino acid sequence are configured to form a coiled coil. The second alpha helix forming amino acid sequence and the fourth alpha helix forming amino acid sequence are configured to form a coiled coil.

A method for analyte detection is provided herein. The method includes mixing a sample containing an analyte with a test solution, the test solution comprising: a first polypeptide comprising a first alpha helix forming amino acid sequence configured to bind a second alpha helix forming amino acid sequence linked by a first flexible linker peptide; a second polypeptide, comprising a third alpha helix forming amino acid sequence configured to bind a fourth alpha helix forming amino acid sequence linked by a second flexible linker peptide; and a first signal generation component operably linked to the first polypeptide and a second signal generation component operably linked to the second polypeptide and a first analyte binding component operably linked to the first polypeptide and a second analyte binding component operably linked to the second polypeptide configured to promote formation of a dimer upon analyte binding that will persist after dissociation from the analyte. The first alpha helix forming amino acid sequence and the third alpha helix forming amino acid sequence are configured to form a coiled coil. The second alpha helix forming amino acid sequence and the fourth alpha helix forming amino acid sequence are configured to form a coiled coil; and detecting a signal.

The foregoing broadly outlines the features and technical advantages of the present disclosure in order that the detailed description that follows may be better understood. Additional features and advantages of the disclosure will be described hereinafter that form the subject of the claims of this application. It will be appreciated by those of skill in the art that the conception and specific aspects disclosed herein may be readily utilized as a basis for modifying or designing other aspects for carrying out the same purposes of the present disclosure within the spirit and scope of the disclosure and provided in the appended claims.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

A detailed description of the invention is hereafter provided with specific reference being made to the drawings in which:

FIG. 1A shows the interactions between two alpha helices bound to form a coiled coil.

FIG. 1B shows the effects of various substitutions on binding free energy.

FIG. 2A shows the hairpin structure caused by intramolecular alpha helix binding of various embodiments.

FIG. 2B shows the possible conformations and intramolecular binding interactions of various embodiments.

FIG. 3A shows an example of a polypeptide with peptide sequence SEQ ID NO: 42.

FIG. 3B shows an example of a polypeptide with peptide sequence SEQ ID NO: 43.

FIG. 3C shows an example of a dimer formed from the polypeptides in FIGS. 2A and 2B with peptide sequences SEQ ID NO: 42 and 43.

FIG. 3D shows an example of a polypeptide with peptide sequence SEQ ID NO: 44.

FIG. 3E shows an example of a polypeptide with peptide sequence SEQ ID NO: 45.

FIG. 3F shows an example of a dimer formed from the polypeptides in FIGS. 2D and 2E with peptide sequences SEQ ID NO: 44 and 45.

FIG. 4A shows an example of a biosensor of several embodiments.

FIG. 4B shows an example detection of an analyte of several embodiments.

FIG. 4C shows an example of biosensor signaling enhancement of several embodiments.

FIG. 5 shows the results of a theoretical example system of polypeptides consisting of a hairpin folding Gibbs Free Energy of Binding −15 kJ/mol and an analyte concentration of approximately 1 fM based on computational modeling of the system.

FIG. 6 shows the predicted signal produced by an example system of polypeptides consisting of a hairpin folding Gibbs Free Energy of −15 kJ/mol and an analyte concentration ranging from 1 pM to 200 pM based on computational modeling of the system.

FIG. 7A shows an embodiment of a first and second construct. FIG. 7A) Schematic of construct domain structure consisting of the AP or EX fragment of the split APEX gene, two leucine zipper alpha helices and a His tag. Constructs are shown with the N-terminus to the left, although the orientation of the dimer will place the AP and EX portions next to each other.

FIG. 7B shows initial reconstitution assays. Tube 1: AP construct alone, 2: EX construct alone, 3: AP construct+EX construct. Samples were mixed with 3 μM Heme and incubated for 10 minutes. 3,3′,5,5′-tetramethylbenzidine (TMB) substrate solution was then added and the solution incubated for 10 minutes.

### DETAILED DESCRIPTION

Various aspects are described below with reference to the drawings. The relationship and functioning of the various elements of the aspects may better be understood by reference to the following detailed description. However, aspects are not limited to those illustrated in the drawings or explicitly described below. It should be understood that the drawings are not necessarily to scale, and in certain instances, details may have been omitted that are not necessary for an understanding of aspects disclosed herein, such as conventional fabrication and assembly. Headings are provided for the convenience of the reader and to assist organization of the disclosure and should not be construed to limit or otherwise define the scope of the invention.

The terms “polypeptide”, “peptide”, “peptide sequence”, “protein” and “protein sequence” are used interchangeably in this disclosure. They refer to a polymeric form of amino acids of any length, or analogs thereof. Polypeptides may have any three-dimensional structure, and may perform any function, known or unknown. A polypeptide may comprise one or more modified amino acids. If present, modifications to the amino-acid structure may be imparted before or after assembly of the polymer. The sequence of amino acids may be modified by non-protein components. A polypeptide may be further modified after polymerization, such as by conjugation with a labeling component.

The term “recombinant” is understood to mean that a particular nucleic acid (DNA or RNA) or protein is the product of various combinations of cloning, restriction, or ligation steps resulting in a construct having a structural coding or non-coding sequence distinguishable from endogenous nucleic acids found in natural systems.

The terms “construct”, “cassette”, or “expression cassette” is understood to mean a recombinant nucleic acid, generally recombinant DNA, which has been generated for the purpose of the expression or propagation of a nucleotide sequence(s) of interest, or is to be used in the construction of other recombinant nucleotide sequences.

The term “promoter” is understood to mean a regulatory sequence/element or control sequence/element that is capable of binding/recruiting a RNA polymerase and initiating transcription of sequence downstream or in a 3′ direction from the promoter. A promoter can be, for example, constitutively active or always on or inducible in which the promoter is active or inactive in the presence of an external stimulus. An example of a promoter is a T7 promoter.

The term “operably linked” can mean the positioning of components in a relationship which permits them to function in their intended manner For example, a promoter can be linked to a polynucleotide sequence to induce transcription of the polynucleotide sequence.

The term “percent identity” refers to the number of identical amino acid residues over a defined length of a given alignment (e.g., 4, 5, and 6 out of 6 being 66.67%, 83.33%, and 100% identical). “Substantially identical” as used herein refers to a degree of identity that is at least 40%, 50%, 60%, 62.5%, 70%, 75%, 80%, 85%, 90%, 95%. 97%, 98%, 99%, or 100%, or percentages in between over a region of amino acids. “Percent similarity” refers to the number of physiochemically similar amino residues over a defined length of a given alignment (e.g., 4, 5, and 6 out of 6 being 66.67%, 83.33%, and 100% identical), allowing for substitution of similar amino acids. For example, the hydrophobic amino acid Leucine would be similar to the amino acids Isoleucine and Valine. “Substantially similar” as used herein refers to a degree of similar amino acids that is at least 40%, 50%, 60%, 62.5%, 70%, 75%, 80%, 85%, 90%, 95%. 97%, 98%, 99%, or 100%, or percentages in between over a region of amino acids.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Examples of conservative mutations include amino acid substitutions of amino acids within the sub-groups above, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained. Exemplary conservative amino acid substitutions are shown in the following chart:

| Type of Amino Acid | Substitutable Amino Acids |
|---|---|
| Hydrophilic | Ala, Pro, Gly, Glu, Asp, Gln, |
| | Asn, Ser, Thr |
| Acidic (Negatively charged at pH = 7) | Asn and Glu |
| Sulphydryl | Cys |
| Aliphatic | Val, Ile, Leu, Met |
| Basic (Positively chared at pH = 7) | Lys, Arg, His |
| Aromatic | Phe, Tyr, Trp |

The term “signal” refers to any measurable indication of binding. A non-limiting list of examples includes generation of colored products, fluorescence, or electrical current, as well as shifts in or destruction of colored products, fluorescence, or electrical current. “Signal amplification” or “amplification of the signal” refers to an increase in the amount of signal above the amount expected for a given concentration of analyte Amplification can be linear or non-linear. A non-limiting list of examples include catalysis of multiple substrate molecules by an enzyme over time, the generation of multiple signaling complexes from a single analyte, and the inclusion of sequential reactions. Multiple forms of amplification can be combined.

To address the limitations of detecting analyte in a cell-free environment using split enzymes, a system is provided that regulates dimerization to favor reconstitution of the enzyme only when bound to the analyte. The present invention allows for amplification of the signal in a single vessel reaction, which would allow split enzymes methods to expand beyond cells into analyte detection methods with broad applications.

The disclosure generally relates to compositions, polypeptides, methods, and systems for controlling protein dimerization or oligomerization of biosensors using intramolecular to intermolecular conformational switches.

In various embodiments are disclosed polypeptides comprising two amphiphilic alpha-helices comprising 14 to 49 amino acids connected by a flexible peptide sequence comprising 2 to 20 amino acids and configured to form an anti-parallel, intramolecular coiled coil. In various embodiments, the alpha helices contain amino acids configured to bind using hydrophobic interactions. In various embodiments, the alpha helices contain acidic and basic amino acids configured to create salt-bridge interactions with the other alpha helix.

In various embodiments, the amino acids configured to bind using hydrophobic interactions are selected from alanine, valine, leucine, and isoleucine. In various embodiments, the acidic amino acids configured to create salt-bridge interactions are selected from aspartate and glutamate and the basic amino acids are chosen from Arginine, Lysine, and Histidine.

In various embodiments, the number of hydrophobic interactions between the two linked alpha helices is chosen to achieve a desired Gibbs free energy of binding. In various embodiments the number of salt-bridge interactions between the two linked alpha helices is chosen to achieve a desired Gibbs free energy of binding. In various embodiments the number of hydrophobic interactions and salt-bridge interactions between the two linked alpha helices are chosen together to achieve a desired Gibbs free energy of binding.

In some embodiments, the first alpha helix forming amino acid sequence and the third alpha helix forming amino acid sequence form a coiled coil that is more energetically favorable than an alpha helix formed between the first alpha helix forming amino acid sequence and the second alpha helix forming amino acid sequence.

In some embodiments, the second alpha helix forming amino acid sequence and the fourth alpha helix forming amino acid sequence form coiled coil that is more energetically favorable than an alpha helix formed between the second alpha helix forming amino acid sequence and the first alpha helix forming amino acid sequence.

As used herein “energetically favorable” refers to interactions that are more stable or yields interactions of higher affinity.

In various embodiments, the number of hydrophobic interactions is controlled by the length of the alpha helices. In various embodiments the number of hydrophobic interactions is controlled by replacing hydrophobic amino acids along the binding surface with hydrophilic amino acids. In various embodiments, the hydrophilic amino acids along the binding surface help dictate orientation of the coiled coil.

In various embodiments, the number of salt bridges is controlled by the length of the alpha helices. In various embodiments, the number of salt bridges is controlled by replacing acidic amino acids with polar amino acids. In various embodiments, the number of salt bridges is controlled by replacing basic amino acids with polar amino acids. In various embodiments, the number of salt bridges is controlled by replacing basic amino acids with acidic amino acids to create a repulsive charge interaction. In various embodiments, the number of salt bridges is controlled by replacing acidic amino acids with basic amino acids to create a repulsive charge interaction.

In various embodiments, the polypeptide is configured to form an intramolecular covalent bond. In various embodiments, this covalent bond is a disulfide bond between cysteines within the alpha helices. In various embodiments, this covalent bond is a disulfide bond between cysteines within the first flexible peptide linker and the second flexible peptide linker. In various embodiments, this covalent bond is a disulfide bond between cysteines upstream or downstream of the coiled coil.

In various embodiments are disclosed a system containing two or more polypeptides as described above further configured to bind through intermolecular hydrophobic and salt-bridge interactions. In various embodiments, the intermolecular interaction has a more favorable Gibbs free energy of binding, resulting in longer binding half-lives.

In various embodiments, the polypeptides are configured to form an intermolecular covalent bond. In various embodiments, this covalent bond is a disulfide bond between cysteines within the alpha helix. In various embodiments, this covalent bond is a disulfide bond between cysteines within the first flexible peptide linker and the second flexible peptide linker. In various embodiments, this covalent bond is a disulfide bond between cysteines upstream or downstream of the coiled coil.

In various embodiments are disclosed systems of two or more polypeptides of various embodiments configured to non-competitively bind to an analyte. In various embodiments, the two or more polypeptides of various embodiments bind to nearby analytes.

In various embodiments are disclosed systems of polypeptides comprising of two or more of the coiled coil motifs (alpha helix forming amino acid sequences) described above operably linked to a component of a split enzyme configured so reconstitution of the complete enzyme is facilitated by the intermolecular binding conformation.

In some embodiments, the system further includes a first signal generation component operably linked to the first polypeptide and a second signal generation component operably linked to the second polypeptide and a first analyte binding component operably linked to the first polypeptide.

In some embodiments, the first signal generation component and the second signal generation component are subunits of a split enzyme.

In some embodiments, the split enzyme is a fluorescent protein, a peroxidase, a bioluminescence generating enzyme, a FRET pair, or a multimeric enzyme complex.

In various embodiments, the split enzyme is selected from split-GFP, split-HRP, split-APEX2, split-Luciferase, and split-betagalactosidase.

In various embodiments are disclosed systems of polypeptides comprising of two or more of the coiled coil motifs described above operably linked to a component of a multimeric enzyme complex configured so enzyme activity is facilitated by the intermolecular binding conformation. An example of a multimeric enzyme system is BLA-BLIP. Another example is AP-GOx.

In various embodiments are disclosed systems of polypeptides comprising of two or more of the alpha helix forming amino acid sequences described above operably linked to a component of a Fluorescence Resonance Energy Transfer (FRET) system configured so reconstitution of the fluorescence emission of the complex is changed by the intermolecular binding conformation. In various embodiments, the FRET pair is selected from EBFP2-mEGFP, ECFP-EYFP, Cerulean-Venus, MiCy-mKO, TFP1-mVenus, CyPet-YPet, EGFP-mCherry, Venus-mCherry, Venus-tdTomato, Venus-mPlum, Fluorescein-BHQ, and Rhodamine-Dabcyl.

In various embodiments, the conformational switch is measured by color change of an enzymatic substrate. In various embodiments, the conformational switch is measured by light detection. In various embodiments, the conformational switch is measured by fluorescence. In various embodiments, the conformational switch is measured by electrical current detection.

In various embodiments are disclosed expression cassettes, plasmids, vectors, or expression vectors including a polynucleotide coding for the polypeptide of various embodiments and a promoter polynucleotide operably linked to the polypeptide of various embodiments, wherein the promoter polynucleotide is recognized by an RNA polymerase and is capable of directing the RNA polymerase to transcribe the polynucleotide coding for the polypeptide of various embodiments.

In various embodiments the polypeptides described above are synthesized in bacterial cells. In various embodiment the polypeptides are synthesized in eukaryotic cells. In various embodiments, the polypeptides are synthesized in vitro.

In various embodiments, the rate of the conformational switch from the intramolecular conformation to the intermolecular conformation is controlled by the length of the flexible polypeptide linker. In various embodiments, the rate of the conformational switch is controlled by the pH of the reaction buffer. In various embodiments, the rate of the conformational switch is controlled by the ionic strength of the reaction buffer. In various embodiments, the rate of the conformational switch is controlled by reaction temperature.

In various embodiments are disclosed methods and systems for detecting an analyte of choice, the methods and systems including the steps of: collecting a sample, creation of buffer and reaction conditions of various embodiments, addition of the polypeptides of various embodiments, and detection of the signal. In various embodiments are disclosed polypeptides comprising two amphiphilic alpha-helices comprising 14 to 49 amino acids connected by a flexible peptide sequence comprising 2 to 20 amino acids and configured to form a parallel or anti-parallel, intramolecular coiled coil. In various embodiments, the alpha helices contain amino acids configured to bind using hydrophobic interactions. In various embodiments, the alpha helices contain acidic and basic amino acids configured to create salt-bridge interactions with the other alpha helix.

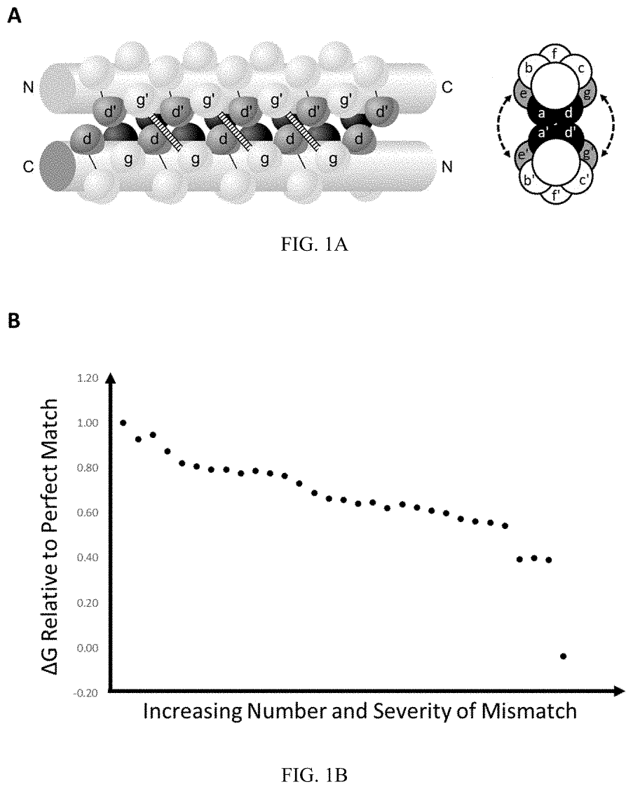

As shown in FIG. 1A, amphiphilic alpha helices containing hydrophobic amino acids along one side flanked by charged amino acids can dimerize in either a parallel or antiparallel orientation. This binding is driven by both the hydrophobic-hydrophobic and salt bridge interactions between the two motifs.

The simple structure and content of this motif allows the affinity and stability of the bound alpha helices to be predicted with a high level of accuracy, as shown in FIG. 1B, aiding their design and implementation in synthetic systems. For example, the number and strength of hydrophobic interactions between the two alpha helices can be controlled by substituting hydrophobic amino acids for hydrophilic amino acids or other hydrophobic amino acids. Likewise, the number of salt-bridge interactions, which form between amino acids with opposite charges, can be controlled by substituting one or both amino acids in the salt-bridge with polar or amino acids with a charge that will result in a repulsive interaction.

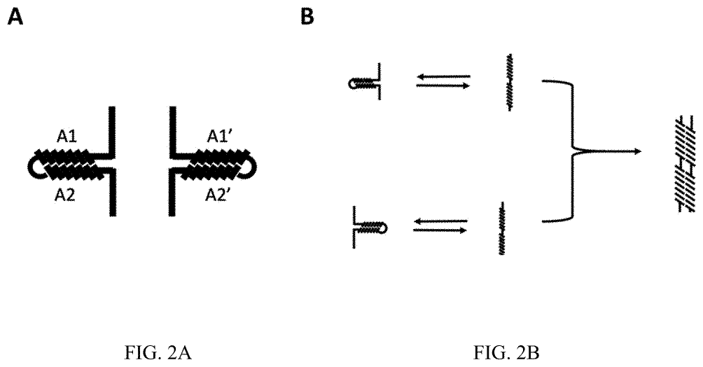

As shown in FIG. 2A, these two alpha helices can be operably linked to form an anti-parallel coiled coil. Sequences A1, A2, A1′, and A2′ correspond to SEQ ID NOs: 1, 2, 3, and 4, respectively, The ratio of time spent in the folded and unfolded states (K_{f}) is dependent on the Gibbs free energy of the intramolecular dimer as described in the following equation:

K_{f}=e^{-ΔG/RT}

where ΔG is the Gibbs free energy of folding, R is the gas constant, and T is the temperature in Kelvin.

Thus, by regulating the amino acid content of the alpha helices, the ratio of folded to unfolded protein can be controlled.

In various embodiments, the amino acid sequences of the polypeptide is substantially similar to SEQ ID NOs: 42-73.

In various embodiments are disclosed a system containing two or more polypeptides as described above further configured to bind through intermolecular hydrophobic and salt-bridge interactions.

As shown in FIG. 2B, two complimentary polypeptides containing the alpha-helical motif described above can be designed to also form intermolecular binding interactions. This binding can result from random interactions of the proteins in the open conformation or by an induced conformational change due to interactions between the proteins. In the case of the first model, the rate of dimer formation is a function of the Gibbs free energy of folding and the concentration of the monomers:

V=kₒₙ[A](e^{ΔG}^{_{A}}^{/RT})[B](e^{ΔG}^{_{B}}^{/RT})−k_{off}[AB]

where V is the rate of binding, [A] is the concentration of the first polypeptide, [B] is the concentration of the second polypeptide, kₒₙ is the association rate of the polypeptides, k_{off} is the dissociation rate of the complex, ΔG_{A} is the Gibbs free energy of folding for the first polypeptide, ΔG_{B} is the Gibbs free energy of folding for the second polypeptide, R is the gas constant, and T is the temperature in Kelvin.

Thus, the kinetics of binding for a given set of polypeptides at a given concentration is dependent on their ΔG of folding, which can be controlled. This allows the rate of intermolecular binding to be controlled by creating amino acid substitutions in one or both polypeptides.

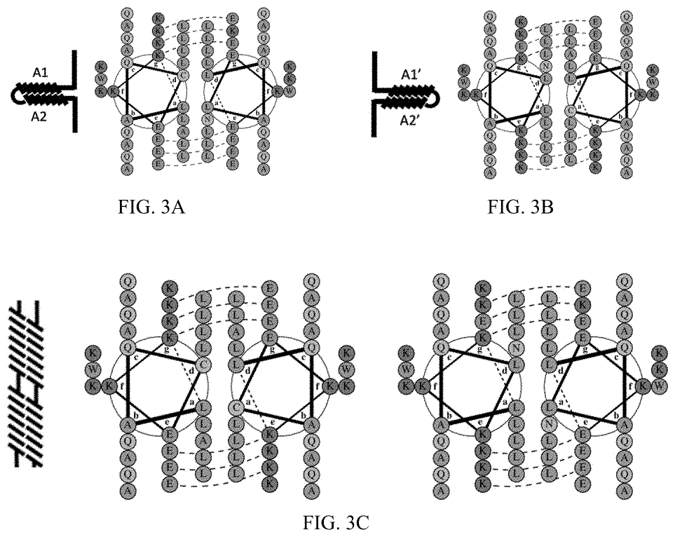

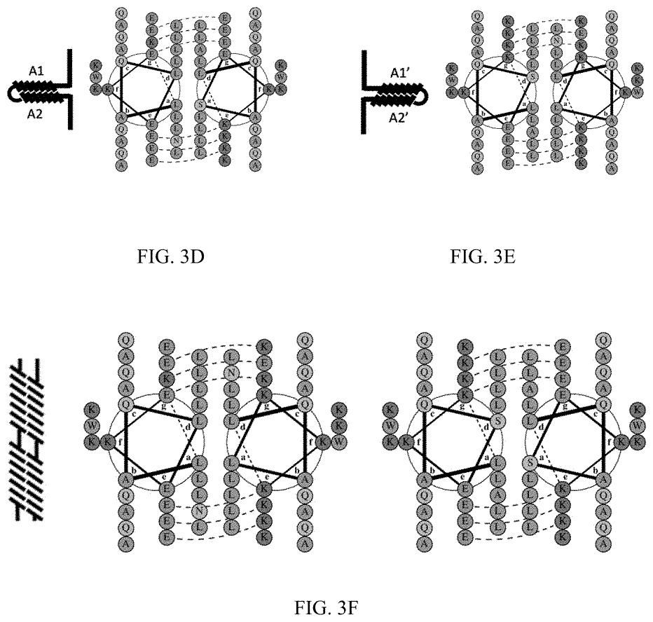

In various embodiments, the intermolecular interactions are configured to be more stable than the intramolecular interactions. For example, FIG. 3A shows the intramolecular interactions of SEQ ID NO: 42 containing two repulsive salt-bridge interactions and two hydrophilic amino acids interacting with hydrophobic amino acids. FIG. 3B shows a complimentary polypeptide, SEQ ID NO: 43, that also has two repulsive salt-bridge interactions and two hydrophilic amino acids interacting with hydrophobic amino acids. This will result in a moderately unstable folding conformation. However, as shown in FIG. 3C, the intermolecular conformation results in no mismatched hydrophobic amino acids and the maximum number of salt-bridge interactions.

A second example is shown in FIGS. 3D-3F, where each polypeptide contains four repulsive salt-bridge interactions, two hydrophilic amino acids interacting with hydrophobic amino acids, and an alanine interacting only weakly with a corresponding Leucine. However, again the resulting dimer has a more stable interaction due to the increased number of interactions overall, the alignment of hydrophilic amino acids and the removal of repulsive salt-bridge interactions.

Because the second example contains fewer salt-bridges than the first example, it is expected to have a lower K_{f}, which leads to more proteins in the open conformation and, as a result, a higher rate of intermolecular binding. However, both will produce very stable intermolecular complexes. Thus, in this example the rate of binding is specifically controlled without affecting the equilibrium binding characteristics of this system.

In one embodiment, the complimentary pair of polypeptides consists of SEQ ID NO: 42 and SEQ ID NO: 43.

In one embodiment, the complimentary pair of polypeptides consists of SEQ ID NO: 44 and SEQ ID NO: 45.

In one embodiment, the complimentary pair of polypeptides consists of SEQ ID NO: 46 and SEQ ID NO: 52.

In one embodiment, the complimentary pair of polypeptides consists of SEQ ID NO: 47 and SEQ ID NO: 53.

In one embodiment, the complimentary pair of polypeptides consists of SEQ ID NO: 48 and SEQ ID NO: 54.

In one embodiment, the complimentary pair of polypeptides consists of SEQ ID NO: 49 and SEQ ID NO: 55.

In one embodiment, the complimentary pair of polypeptides consists of SEQ ID NO: 50 and SEQ ID NO: 56.

In one embodiment, the complimentary pair of polypeptides consists of SEQ ID NO: 51 and SEQ ID NO: 57.

In one embodiment, the complimentary pair of polypeptides consists of SEQ ID 58 and SEQ ID NO: 66.

In one embodiment, the complimentary pair of polypeptides consists of SEQ ID NO: 59 and SEQ ID NO: 67.

In one embodiment, the complimentary pair of polypeptides consists of SEQ ID NO: 60 and SEQ ID NO: 68.

In one embodiment, the complimentary pair of polypeptides consists of SEQ ID NO: 61 and SEQ ID NO: 69.

In one embodiment, the complimentary pair of polypeptides consists of SEQ ID NO: 62 and SEQ ID NO: 70.

In one embodiment, the complimentary pair of polypeptides consists of SEQ ID NO: 63 and SEQ ID NO: 71.

In one embodiment, the complimentary pair of polypeptides consists of SEQ ID NO: 64 and SEQ ID NO: 72.

In one embodiment, the complimentary pair of polypeptides consists of SEQ ID NO: 65 and SEQ ID NO: 73.

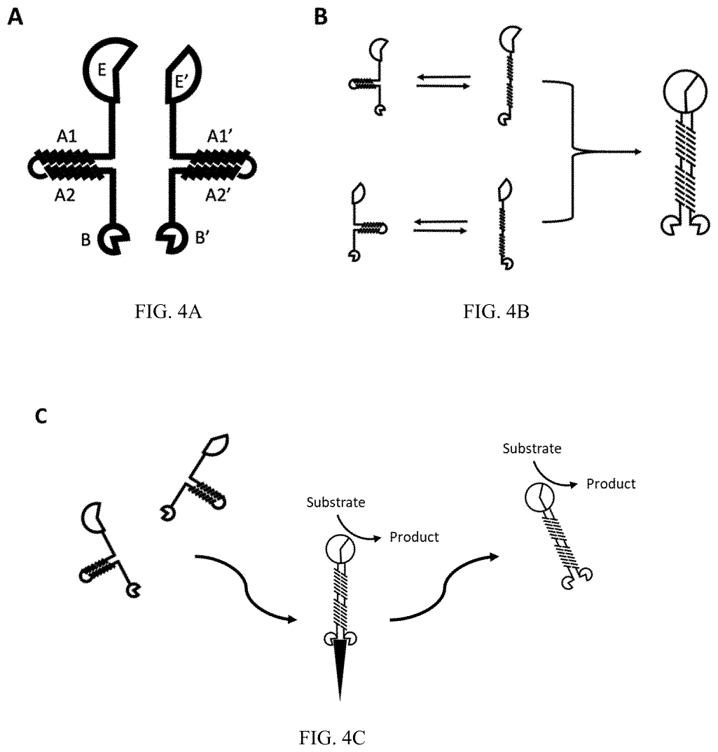

In various embodiments is disclosed a biosensor containing a complimentary pair of polypeptides as described above further operably linked to a signal generating component and an analyte binding component, as shown in FIG. 4A. The polypeptides are said to be complimentary because they can undergo a conformational switch to form two intermolecular coiled coils, as shown in FIG. 4B. This configuration allows the signal generating component to generate signal upon binding to the analyte. Analyte binding also increases the concentration of the two polypeptides, resulting in an increased rate of conformational switching to form an intermolecular interaction between the alpha helices in the two polypeptides, stabilizing the complex. The intermolecular interactions may result in parallel or antiparallel alpha helices. If the half-life of binding between the analyte binding components of the biosensor and the analyte is less than the half-life of the biosensor dimer, then the signal producing complex will remain active after dissociation from the analyte, as shown in FIG. 4C. This results in amplification of the signal, as additional complexes can form on the same analyte over time.

In various embodiments are disclosed systems of polypeptides comprising two or more of the coiled coil motifs described above operably linked to a component of a split enzyme configured so reconstitution of the complete enzyme is facilitated by the intermolecular binding conformation. Split enzymes divide a single polypeptide enzyme into two or more components. These components are generally designed to bind to reconstitute the enzymatic catalysis activity only at very high effective concentrations, such as binding both halves to an analyte. Thus, the components often have low affinity for each other and disassemble upon dissociation from the analyte. The polypeptides described here can be configured to create a stable complex that will allow the reconstituted enzyme to remain active after dissociation from the analyte. A non-limiting list of example split-enzymes is split-GFP, split-HRP, split-APEX2, split-Luciferase, and split-betagalactosidase.

In various embodiments are disclosed systems of polypeptides comprising two or more of the coiled coil motifs described above operably linked to a component of a multimeric enzyme complex configured so enzyme activity is facilitated by the intermolecular binding conformation. Multimeric enzyme complexes can be enzymes requiring the assembly of multiple polypeptides to catalyze a single reaction or complex of enzymes that catalyze subsequent steps in a multistep reaction. In this case, close proximity allows the product of the first enzyme to be rapidly captured by the second enzyme, increasing the overall rate of reaction. In another model, analyte binding can bring an inhibitor in close proximity of an enzyme to slow the rate of the substrate reaction. An example of a multimeric enzyme system is BLA-BLIP. Another example is AP-GOx.

In various embodiments are disclosed systems of polypeptides comprising of two or more of the coiled coil motifs described above operably linked to a component of a Fluorescence Resonance Energy Transfer (FRET) system configured so reconstitution of the fluorescence emission of the complex is changed by the intermolecular binding conformation. A FRET signal occurs when a pair of fluorescent molecules are held in close proximity A pair of fluorescent molecules is complimentary if the excitation wavelength of one, the acceptor, is substantially close to or is equal to the excitation wavelength of the other, the donor. When this condition is met, excitation of the donor with the appropriate wavelength of light results in a fluorescent emission of light that is absorbed by the acceptor molecule, resulting in an emission of light at the acceptor molecule's emission wavelength. The ratio of light emission at the donor and acceptor molecules' emission wavelengths can be used to measure the amount and rate of binding. In a variation of FRET, the acceptor probe is a molecule capable of absorbing light at the emission wavelength of the donor but does not produce detectable fluorescence emission (called a quencher). A non-limiting list of FRET pairs is EBFP2-mEGFP, ECFP-EYFP, Cerulean-Venus, MiCy-mKO, TFP1-mVenus, CyPet-YPet, EGFP-mCherry, Venus-mCherry, Venus-tdTomato, Venus-mPlum, Fluorescein-BHQ, and Rhodamine-Dabcyl.

In various embodiments, the analyte binding domain is selected from an ScFV, a Fa b, an antibody, a natural analyte binding domain, and a synthetic analyte binding domain. ScFVs and Fab fragments are derived from portions of an antibody containing the antigen binding domain. These can be designed to bind multiple epitopes on a single analyte or on nearby analytes, such as two components of a complex.

In some embodiments, the ScFV is an amino acid sequence having at least 70% sequence identity to an amino acid sequence selected from the group consisting of: SEQ ID NO: 74, SEQ ID NO: 75, SEQ ID NO: 76, and SEQ ID NO: 77.

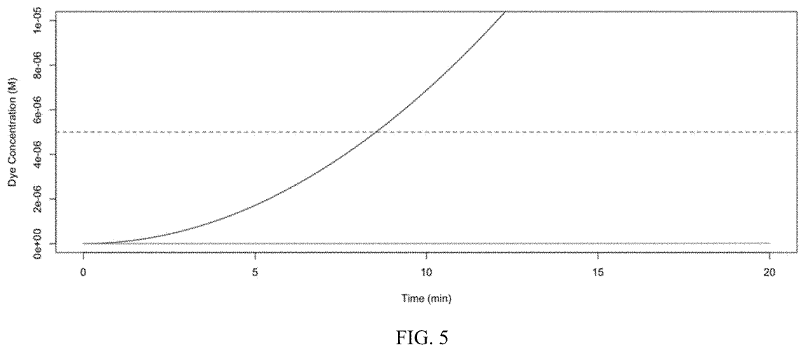

In some embodiments is disclosed a biosensor. Simultaneous analyte binding by both biosensor polypeptides will increase the rate of conformational switching to the intermolecular interactions. This rate of increase can be due to either an induced change or by increasing their effective concentration due to their close proximity and fixed orientation on the analyte. The customizability of the amino acid sequences in the alpha helices allow the rate to be controlled such that the difference between the rate on the analyte and the rate in solution is easily measured. To demonstrate the feasibility of this biosensor, modeling of the kinetics of this system was conducted assuming a biosensor with the following parameters: ΔG of folding for both intramolecular coiled coils of −15 kJ/mol, ScFV for analyte binding with a kD of 100 nM, split APEX2 signal generating component and TMB substrate. As shown in FIG. 5, samples containing 1.7 fM analyte concentrations were visually discernable in approximately 8 minutes, while a sample without analyte was undetectable even at 20 minutes of incubation.

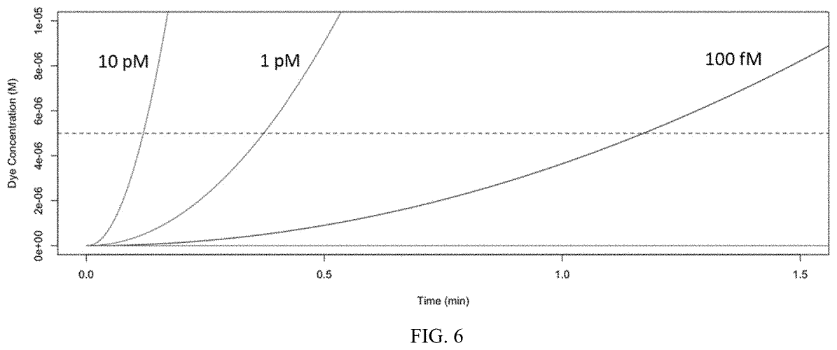

The amount of signal produced can also be measured to determine the amount of analyte in solution quantitatively. As shown in FIG. 6, the rate of signal production is proportional to the concentration of the analyte in solution. Thus, measurements of the rate of signal production using various methods could be used to determine the amount of analyte in the sample.

An example application of this biosensor is for qualitative detection of viruses or bacteria. The rapid, inexpensive, and sensitive detection of analytes in biological or environmental samples would greatly improve health and safety. For example, the detection of viral or bacterial infections at home or the point of care would make diagnosis more accurate and rapid, while limiting exposure to others. Rapid and inexpensive tests would also allow frequent testing of water supplies, restaurant surfaces, and other potential sources of exposure to further decrease the spread of existing or emerging diseases. However, current methods are limited due to the time, expertise, lack of sensitivity, and special equipment they may require, leading to high costs and slow turnaround times.

An example of a protocol for pathogen detection is:

1. Open a plastic tube that contains both polypeptides of a biosensor lyophilized inside.
2. Rehydrate and prepare the reaction by adding 200 ul of buffer containing substrate.
3. Swab the patient or surface being tested and insert the swab into the tube.
4. Close the lid and mix by inverting several times.
5. Incubate at room temperature for 15 minutes.
6. Visually inspect and compare the solution color to a provided color scale.

Another example application of this biosensor is the quantitative detection of insulin from blood. Type 2 Diabetes is a growing health concern in the United States and worldwide. Early detection of the disease requires the detection of elevated blood serum insulin levels. However, there are currently no widely available, inexpensive, and rapid tests for insulin levels. Creation of a method that requires minimal expertise, time, and equipment to allow the point of care measurement of insulin would greatly increase access to early diagnosis and treatment of Type 2 Diabetes. An example protocol for insulin detection is:

1. Collect 200 ul of blood from a patient in a capillary tube.
2. Place the sample on a microfluidic device configured to separate the serum and to mix the sample with a biosensor configured to recognize insulin and to generate a FRET signal.
3. Insert microfluidic device into a fluorescence reader.
4. Read FRET signal change over time to determine insulin concentration.
In some embodiments, the biosensor is a pair of amino acid sequences selected from the group consisting of: SEQ ID NO: 78, SEQ ID NO: 79, SEQ ID NO: 80, SEQ ID NO: 81.

In some aspects, the first alpha helix forming amino acid sequence or the second alpha helix forming amino acid sequence can be SEQ ID NO: 12 and SEQ ID NO: 13, respectively. In some aspects, the first alpha helix forming amino acid sequence or the second alpha helix forming amino acid sequence can be SEQ ID NO: 14 and SEQ ID NO: 15, respectively. In some aspects, the first alpha helix forming amino acid sequence or the second alpha helix forming amino acid sequence can be SEQ ID NO: 16 and SEQ ID NO: 17, respectively. In some aspects, the first alpha helix forming amino acid sequence or the second alpha helix forming amino acid sequence can be SEQ ID NO: 18 and SEQ ID NO: 19, respectively. In some aspects, the first alpha helix forming amino acid sequence can any sequence of SEQ ID NOs: 12-19. In some aspects, the second alpha helix forming amino acid sequence can include any sequence of from SEQ ID NOs: 12-19.

In some aspects, the protein can be any one of SEQ ID NOs: 1-11.

Examples of first and second constructs include, but are not limited to, SEQ ID NOs: 24-41.

In some embodiments, the first alpha helix forming amino acid sequence is an amino acid sequence selected from the group consisting of: SEQ ID NO: 12, SEQ ID NO: 13, SEQ ID NO: 14, SEQ ID NO: 15, SEQ ID NO: 16, SEQ ID NO: 17, SEQ ID NO: 18, and SEQ ID NO: 19.

In some embodiments, the second alpha helix forming amino acid sequence is an amino acid sequence selected from the group consisting of: SEQ ID NO: 12, SEQ ID NO: 13, SEQ ID NO: 14, SEQ ID NO: 15, SEQ ID NO: 16, SEQ ID NO: 17, SEQ ID NO: 18, and SEQ ID NO: 19.

In some embodiments, the third alpha helix forming amino acid sequence is an amino acid sequence selected from the group consisting of: SEQ ID NO: 12, SEQ ID NO: 13, SEQ ID NO: 14, SEQ ID NO: 15, SEQ ID NO: 16, SEQ ID NO: 17, SEQ ID NO: 18, and SEQ ID NO: 19.

In some embodiments, the fourth alpha helix forming amino acid sequence is an amino acid sequence selected from the group consisting of: SEQ ID NO: 12, SEQ ID NO: 13, SEQ ID NO: 14, SEQ ID NO: 15, SEQ ID NO: 16, SEQ ID NO: 17, SEQ ID NO: 18, and SEQ ID NO: 19.

In some embodiments, the first polypeptide is an amino acid sequence having at least 70% sequence identity to an amino acid sequence selected from the group consisting of: SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73.

In some embodiments, the second polypeptide is an amino acid sequence having at least 70% sequence identity to an amino acid sequence selected from the group consisting of: SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73.

In some embodiments, the first polypeptide is an amino acid sequence selected from the group consisting of: SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73.

In some embodiments, the second polypeptide is an amino acid sequence selected from the group consisting of: SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73.

This mechanism allows multiple aspects of protein dimerization to be tightly controlled in several ways. First, the amino acid sequences of the intramolecular alpha helix forming amino acid sequence pair can be modified to control the affinity of the intramolecular dimer. Likewise, the affinity of the intermolecular dimer can be controlled by changing the number and character of the intermolecular interactions. Example designs with differing numbers of interactions are shown in FIG. 2. In addition to changing the amino acid sequence, several buffer parameters, including ionic strength, pH, temperature, presence of detergents and blocking proteins, and the overall concentration of the two constructs, can be optimized to control dimer formation rate. Thus, this system is tunable to a level not previously seen in current proximity labeling or analyte detection methods. Together, these optimizations will allow each assay based on this technology to carefully control sensitivity, specificity, reaction time, and other aspects of detection.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

The following examples provide and illustrate certain features and/or aspects of the disclosure. The examples should not be construed to limit the disclosure to the particular features or aspects described therein.

### EXAMPLES

### Example 1

To demonstrate a potential use of the system provided herein, we created a system consisting of the split APEX2 peroxidase attached to the alpha helix forming amino acid sequence pair shown in FIGS. 3A-3C. In this configuration, the intramolecular conformation consists of two repulsive and four attractive salt bridges and contains two hydrophilic amino acids in the hydrophobic core. This creates a conformational stability similar to naturally occurring leucine zipper homodimers that form only when bound to DNA and can be successfully competed for by the presence of a more stable heterodimer partner. The intermolecular pair consists of no mismatched salt bridges and aligns the hydrophilic amino acids to create a stable hydrophilic pocket.

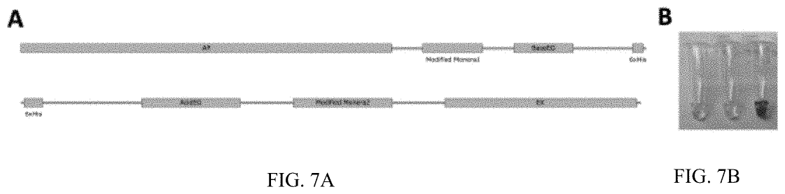

FIG. 7A shows a schematic of the construct domain structures consisting of the AP or EX fragment of the split APEX gene, two alpha helix forming amino acid sequences and a His tag. Constructs are shown with the N-terminus to the left, although the orientation of the dimer will place the AP and EX portions next to each other. FIG. 7B shows Tube 1: AP construct alone, 2: EX construct alone, and 3: AP construct+EX construct. Samples were mixed with 3 uM Heme and incubated for 10 minutes. 3,3′,5,5′-Tetramethylbenzidine (TMB) substrate solution was then added and the solution incubated for 10 minutes. These data demonstrate that the constructs, when at a sufficiently high concentration (approximately 0.5 mg/ml), recombine to reconstitute the active enzyme (FIG. 7B).

The system disclosed herein provides an important tool to detect analytes in solution from a number of sample sources. The constructs can be adapted to target any number of analytes and the amino acid sequences and buffer conditions used can be tuned to the requirements of each assay. These adaptations will allow the system to work within the natural pH, salt, etc. of the sample, which in turn will reduce or eliminate the need for sample pre-processing. The system also enables signal amplification by allowing the reconstituted enzyme to be active after dissociation from the target analyte, increasing the sensitivity of the assay. Together, these innovations enable the development of a wide range of simple, rapid, inexpensive biosensor assays.

Except in the examples, or where otherwise expressly indicated, all numerical quantities in this description indicating amounts of material or conditions of reaction or use are to be understood as modified by the word “about” in describing the broadest scope of the invention. Practice within the numerical limits stated is generally preferred. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which this invention belongs.

Throughout this application, where publications are referenced, the disclosures of these publications in their entireties are hereby incorporated by reference into this application to more fully describe the state of the art to which this invention pertains.

It is also to be understood that this invention is not limited to the specific embodiments and methods described below, as specific components or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “or” is understood to mean “and/or”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

What is claimed is:

1. A system for regulating protein dimerization kinetics comprising:
a first polypeptide comprising a first alpha helix forming amino acid sequence configured to bind a second alpha helix forming amino acid sequence linked by a first flexible linker peptide; and
a second polypeptide comprising a third alpha helix forming amino acid sequence configured to bind a fourth alpha helix forming amino acid sequence linked by a second flexible linker peptide,
wherein the first alpha helix forming amino acid sequence and the third alpha helix forming amino acid sequence are configured to form a coiled coil,
wherein the second alpha helix forming amino acid sequence and the fourth alpha helix forming amino acid sequence are configured to form a coiled coil.

2. The system of claim 1, wherein the first alpha helix forming amino acid sequence and the third alpha helix forming amino acid sequence form a coiled coil that is more energetically favorable than an alpha helix formed between the first alpha helix forming amino acid sequence and the second alpha helix forming amino acid sequence.

3. The system of claim 1, wherein the second alpha helix forming amino acid sequence and the fourth alpha helix forming amino acid sequence form coiled coil that is more energetically favorable than an alpha helix formed between the second alpha helix forming amino acid sequence and the first alpha helix forming amino acid sequence.

4. The system of claim 1, wherein the first alpha helix forming amino acid sequence is an amino acid sequence selected from the group consisting of: SEQ ID NO: 12, SEQ ID NO: 13, SEQ ID NO: 14, SEQ ID NO: 15, SEQ ID NO: 16, SEQ ID NO: 17, SEQ ID NO: 18, and SEQ ID NO: 19.

5. The system of claim 1, wherein the second alpha helix forming amino acid sequence is an amino acid sequence selected from the group consisting of: SEQ ID NO: 12, SEQ ID NO: 13, SEQ ID NO: 14, SEQ ID NO: 15, SEQ ID NO: 16, SEQ ID NO: 17, SEQ ID NO: 18, and SEQ ID NO: 19.

6. The system of claim 1, wherein the third alpha helix forming amino acid sequence is an amino acid sequence selected from the group consisting of: SEQ ID NO: 12, SEQ ID NO: 13, SEQ ID NO: 14, SEQ ID NO: 15, SEQ ID NO: 16, SEQ ID NO: 17, SEQ ID NO: 18, and SEQ ID NO: 19.

7. The system of claim 1, wherein the fourth alpha helix forming amino acid sequence is an amino acid sequence selected from the group consisting of: SEQ ID NO: 12, SEQ ID NO: 13, SEQ ID NO: 14, SEQ ID NO: 15, SEQ ID NO: 16, SEQ ID NO: 17, SEQ ID NO: 18, and SEQ ID NO: 19.

8. The system of claim 1, wherein the first polypeptide is an amino acid sequence having at least 70% sequence identity to an amino acid sequence selected from the group consisting of: SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73.

9. The system of claim 1, wherein the second polypeptide is an amino acid sequence having at least 70% sequence identity to an amino acid sequence selected from the group consisting of: SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73.

10. The system of claim 1, wherein the first polypeptide is an amino acid sequence selected from the group consisting of: SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73.

11. The system of claim 1, wherein the second polypeptide is an amino acid sequence selected from the group consisting of: SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73.

12. The system of claim 1, further comprising a first signal generation component operably linked to the first polypeptide and a second signal generation component operably linked to the second polypeptide and a first analyte binding component operably linked to the first polypeptide.

13. The system of claim 12, wherein the first signal generation component and the second signal generation component are subunits of a split enzyme.

14. The system of claim 13, wherein the split enzyme is a fluorescent protein, a peroxidase, a bioluminescence generating enzyme, a FRET pair, or a multimeric enzyme complex.

15. A biosensor comprising:
a first polypeptide comprising a first alpha helix forming amino acid sequence configured to bind a second alpha helix forming amino acid sequence linked by a first flexible linker peptide,
a second polypeptide comprising a third alpha helix forming amino acid sequence configured to bind a fourth alpha helix forming amino acid sequence linked by a second flexible linker peptide, and
a first signal generation component operably linked to the first polypeptide and a second signal generation component operably linked to the second polypeptide and a first analyte binding component operably linked to the first polypeptide and a second analyte binding component operably linked to the second polypeptide configured to promote formation of a dimer upon analyte binding that will persist after dissociation from the analyte,
wherein the first alpha helix forming amino acid sequence and the third alpha helix forming amino acid sequence are configured to form a coiled coil,
wherein the second alpha helix forming amino acid sequence and the fourth alpha helix forming amino acid sequence are configured to form a coiled coil.

16. The biosensor of claim 15, wherein the first and second signal generation components are configured to only generate signal upon dimerization.

17. The biosensor of claim 15, wherein the first and second signal generation components are subunits of a split enzyme.

18. The biosensor of claim 17, wherein the split enzyme subunits combine to form a fluorescent protein, a peroxidase, a bioluminescence generating enzyme, a FRET pair, or a multimeric enzyme complex.

19. A method for analyte detection comprising:
mixing a sample containing an analyte with a test solution, the test solution comprising:
a first polypeptide comprising a first alpha helix forming amino acid sequence configured to bind a second alpha helix forming amino acid sequence linked by a first flexible linker peptide, and
a second polypeptide, comprising a third alpha helix forming amino acid sequence configured to bind a fourth alpha helix forming amino acid sequence linked by a second flexible linker peptide,
a first signal generation component operably linked to the first polypeptide and a second signal generation component operably linked to the second polypeptide and a first analyte binding component operably linked to the first polypeptide and a second analyte binding component operably linked to the second polypeptide configured to promote formation of a dimer upon analyte binding that will persist after dissociation from the analyte,
wherein the first alpha helix forming amino acid sequence and the third alpha helix forming amino acid sequence are configured to form a coiled coil,
wherein the second alpha helix forming amino acid sequence and the fourth alpha helix forming amino acid sequence are configured to form a coiled coil, and detecting a signal.

20. The method of claim 19, further comprising the addition of buffers to regulate the ionic strength of the reaction.
